(19) United States
(12) Patent Application Publication
(10) Pub. No.: US 2010/0179576 A1
(43) Pub. Date: Jul. 15, 2010

(54) Hernia Repair Kit

(76) Inventor: Nir Halevy, Petach-Tikva (IL)

Correspondence Address: The Law Office of Michael E. Kondoudis, 888 16th Street, N.W., Suite 800, Washington, DC, 20006, US

(21) Appl. No.: 12/634,932

(22) Filed: Dec. 10, 2009

Related U.S. Application Data
(60) Provisional application No. 61/121,578, filed on Dec. 11, 2008.

(51) Int. Cl. A61B 17/03

(52) U.S. Cl. 606/151

(57) ABSTRACT

The present invention provides a hernia repair kit, comprising:

(a) an umbrella-like device comprising a plurality of expandable arms and a mesh mounted to said arms; said umbrella-like device is advanced in a non-expanded state through a trocar and expanded automatically upon release from said trocar;
(b) anchoring elements switchable from a closed configuration in which said anchoring elements are embedded within said mesh, to an open configuration in which said anchoring elements are protruding out of said mesh;
(c) an expandable element characterized by two configurations (i) unexpanded configuration in which said expandable element pushed through said trocar and out of said trocar; (ii) expanded configuration in which said expandable element is expanded upon release from said trocar so as to (i) exert a normal force against said mesh and arms in the direction of said abdominal wall; and, (ii) secure said anchoring elements to said abdominal wall.

### CROSS-REFERENCE TO RELATED APPLICATION

This application claims the benefit of U.S. Provisional Application No. 61/121,578, filed Dec. 11, 2008.

### FIELD OF THE INVENTION

The present invention relates generally to the field of hernia repair. More specifically, the present invention relates to a novel kit for the repair of a hernia that is employed using a laparoscopic procedure.

### BACKGROUND OF THE INVENTION

A hernia occurs when the inside layers of the abdominal muscle have weakened, resulting in a bulge. The abdomen content pushes through the weakened area of the abdominal wall to form a small or large balloon-like sac. This can allow a loop of intestine or organ tissue to push into the sac.

Post Operative Ventral Hernias (POVHs) or Incisional hernias are hernias which form after surgery through the incision site or previous drain sites, or laparoscopic trocar insertion sites. POVH's occur develop in around 2% of patients with clean wounds and 20% in patients with infected wounds following abdominal surgery. The rate is much higher in emergency operations: 50% of POVH's will develop within six months following surgery, while if a hernia does not develop within five years, the chances of the patient developing one are very low. Certain risk factors predispose patients to develop incisional hernias, such as obesity, diabetes, respiratory insufficiency (lung disease), steroids, postoperative wound infection, smoking, inherited disorders such as Marfan's syndrome and Ehlers-Danlos syndrome, as well as poor surgical technique. Approximately 90,000-100,000 incisional hernia repairs are performed annually in the United States.

An inguinal hernia is a protrusion of abdominal-cavity contents through the inguinal canal. Incisional hernias present much the same way inguinal hernias do. That is, they present with a bulge near or at a previous incision. Some patients may experience discomfort, abdominal cramping or complete intestinal obstruction, and subsequent hospitalization as a result of these hernias.

The principle of surgical hernia repair entails the use of prosthetic mesh to repair large defects in order to minimize tension on the repair. A tension free repair has a lesser chance of hernia recurrence. Traditionally, the old scar is incised and removed, and the entire length of the incision inspected. Generally, there are multiple hernia defects other than the one(s) discovered by physical examination. The area requiring coverage is usually large and requires much surgical dissection. A prosthetic mesh is used to cover the defect(s), and the wound closed. This is a major surgical procedure and often complicated. Infection rates following repair may be as high a 7.0%. Recurrence can be up to 5%, or higher, depending on the patient's preoperative risk factors. While the use of prosthetic mesh has decreased the number of recurrences, it has also been implicated in increased infection rates, adhesion or scar formation of the abdominal contents to the anterior abdominal wall leading to intestinal obstruction and fistula formation. However, overall, recovery is usually excellent and patients return to normal activity within a matter of weeks.

The laparoscopic repair of ventral hernias was designed to minimize operative trauma to the patient. As mentioned, these are often complicated repairs requiring large incisions and extensive tissue dissection. In one technique for laparoscopic ventral hernia repair, a large piece of prosthetic mesh is placed under the hernia defect with a wide margin of mesh outside the defect. In one scenario, the mesh is anchored into place with eight full-thickness sutures and secured to the anterior abdominal wall with a varying number of tacks, placed laparoscopically.

In recent years, various hernia repair devices have been developed in order to improve the hernia repair process and reduce the risk of complications or recurrence. U.S. Pat. No. 5,397,331 to Himpens et al. relates to a supporting device and apparatus for inserting the device. The device is inserted in the abdominal cavity through the hernial canal. The device includes a barrier material which may for instance be a synthetic net. The device has a stiffener allowing a controlled deformation of the barrier material such that the device may pass through the guard tube, following which the stiffener is capable of unfolding the barrier material into a substantially planar condition. After insertion and unfolding the device may be tightened against the peritoneum by means of a thread that may be fastened on the surface of the patient's skin.

U.S. Pat. No. 6,669,735 to Pelissier relates to a prosthesis for surgical treatment of hernia. The prosthesis includes two parts, that is, a synthetic non-resorbent mesh and a ring fixed at the peripheral edge of the synthetic mesh. The ring, which is made of a resorbent material, is of a flexibility that permits it to bend, then resume its initial shape to give the mesh a memory of its initial shape. The ring has an interruption intended to be positioned over the femoral veins.

U.S. Pat. No. 5,824,082 to Brown relates to a patch for endoscopic repair of hernias comprising a prosthesis for use in hernia repair surgery having a preformed prosthetic fabric supported along its periphery by shape memory alloy wire having a transformation temperature corresponding to normal body temperature allowing the prosthesis to be tightly rolled into a cylindrical configuration for delivery through a laparoscopic instrument and which deploys to a predetermined shape as it warms up to body temperature.

U.S. Pat. No. 5,425,740 to Hutchinson relates to a clip unit for hernia repair surgery having first and second arms joined at a hinge joint. A latch or hook locks the arms together after they are closed. A mesh material extends around the outside edges of the clip. In use, the clip is placed over a portion of the elevated peritoneum and then closed and locked. The mesh is fixed to the surrounding peritoneum. The clip and surgical method expedites hernia repair surgery and reduces trauma and medical costs.

U.S. Pat. No. 6,517,584 to Lecalve relates to a flexible prosthesis, in particular for curing hernias by colioscopy, the prosthesis including at least one anchor device made of shape memory material designed to be deformed slightly under temperature control from a storage position into a fixing position in which the anchor device interferes with the surrounding tissue.

### SUMMARY OF THE INVENTION

It is the primary goal of the present invention to provide a hernia repair kit for repairing a hernia defect in an efficient, reliable, and relatively quick manner. The repair kit is especially useful for repairing POVH's using the laparoscopic procedure, thus allowing for a shorter hospital stay and less complications when compared to traditional surgical procedures.

The kit of the present invention includes two main components, both of which are designed to be collapsible and insertable though a trocar so as to reach the hernia defect site. The first component is a prosthetic device comprising a mesh material fitted upon an expandable frame. The expandable frame preferably expands umbrella-like so as to adopt a configuration similar to a planar spider web. The prosthetic device may be designed to have any suitable overall shape according to the size, location, and number of which are being repaired. The device should preferably be designed to extend 5-10 cm on the side of the overall hernia defect. The prosthetic device may in some embodiments be formed from a temperature-sensitive material so as to expand upon release into the body.

The prosthetic device also preferably comprises a plurality of attachment elements for attachment of the device to the abdominal wall. The attachment elements may take forms similar, for example, to nails or hooks formed of bio-compatible materials, such attachment elements being well known in the art. After the prosthetic device is inserted though the abdominal wall and spread out, the attachment elements provide a means for securing the prosthetic device to the wall, so as to completely cover the hernia defect.

The prosthetic device is preferably provided inside of a tube having a diameter of about 10 mm, such that it can fit inside of a standard trocar.

The second component of the hernia repair kit comprises an anchoring device. The anchoring device includes a plurality of arms that extend outward radially from a central support rod. The function of the anchoring device is to push the prosthetic device against the abdominal wall, causing the attachment elements of the prosthetic device to become anchored into the wall.

It will be appreciated that the hernia repair kit of the present invention may be adapted for use in fixing other types of hernias as well.

The present invention thus relates to a hernia repair kit comprising;
(a) a prosthetic device comprising an expandable frame and a mesh material mounted on the frame, and further comprising a plurality of attachment elements for facilitating attachment to a body wall; the prosthetic device is adapted for being advanced in a non-expanded state through a hernia defect using a trocar and expanded automatically upon release from the trocar, and;
(b) an anchoring device comprising a plurality of arms, adapted for switching from a closed configuration in which the arms are collapsed inside of the trocar, to an open configuration in which the arms extend outward, for facilitating securing of the prosthetic device to said body wall using the attachment elements.

It will be appreciated that the prosthetic device may comprise any suitable prosthetic device adapted for use in fixing a hernia defect, as are well known in the art. The mesh material may comprise, for example, any suitable patch or other woven element known in the art for use in closing a bodily opening.

Preferably, the arms of the anchoring device extend outward in a substantially radial manner. It is appreciated that other orientations are possible as well.

The present invention also relates to a method for repairing a post operative ventral hernia, comprising;
(a) providing a hernia repair kit, the kit comprising;
   (i) a prosthetic device comprising an expandable frame and a mesh material mounted on the frame, and further comprising a plurality of attachment elements for facilitating attachment to a body wall, wherein the prosthetic device is adapted for being advanced in a non-expanded state through a hernia defect using a trocar and expanded automatically upon release from the trocar, and;
   (ii) an anchoring device comprising a plurality of arms, adapted for switching from a closed configuration in which the arms are collapsed inside of the trocar, to an open configuration in which the arms extend outward, for facilitating securing of the prosthetic device to the body wall using the attachment elements;
(b) insufflating the abdominal wall;
(c) performing dissection of the bowel loops of the hernia;
(d) introducing the prosthetic device to the hernia site using a trocar and opening the prosthetic device inside of the abdominal cavity;
(e) fixing the prosthetic device into place using the anchoring device, and;
(f) optionally, using a mesh patch to seal the hernia opening.

It is appreciated that in the context of the present invention, the term “trocar” is means to refer to any tubular element adapted for use in laparoscopic procedures for allowing advancement of medical elements to the site of the procedure.

It is one object of the present invention to provide a hernia repair kit adapted to repair hernia defect by attachment of a mesh to the abdominal wall, said kit comprising:
(a) an umbrella-like device comprising a plurality of expandable arms 907 and a mesh 906 mounted to said arms 907; wherein said umbrella-like device is adapted for being advanced in a non-expanded state through said hernia defect using a trocar and expanded automatically upon release from said trocar;
(b) anchoring elements 22 at the distal ends of said arms 907, said anchoring elements 22 being adapted for switching from a closed configuration in which said anchoring elements 22 are embedded within said mesh 906, to an open configuration in which said anchoring elements 22 are protruding out of said mesh 906; said anchoring elements being adapted to secure said mesh 906 to said abdominal wall 910;
(c) an expandable element 911 characterized by two configurations (i) unexpanded configuration in which said expandable element 911 is adapted to be pushed through said trocar and out of said trocar; (ii) expanded configuration in which said expandable element 911 is adapted to be expanded upon release from said trocar so as to (i) exert a normal force against said mesh 906 and arms 907 in the direction of said abdominal wall; and, (ii) secure said anchoring elements 22 to said abdominal wall 910.

It is another object of the present invention to provide the hernia repair kit as defined above, wherein said expandable element 911 is a balloon.

It is another object of the present invention to provide the hernia repair kit as defined above, adapted for use in repairing a post operative ventral hernia.

It is another object of the present invention to provide the hernia repair kit as defined above, wherein said anchoring elements comprise elements selected from a group consisting of hooks, staple, tacks or any combination thereof.

It is another object of the present invention to provide the hernia repair kit as defined above, wherein said prosthetic device is formed from material selected from a group consisting of Nitinol or electroactive polymers or any combination thereof.

It is another object of the present invention to provide the hernia repair kit as defined above, wherein arms of said anchoring device extend outward in a substantially radial manner.

It is another object of the present invention to provide the hernia repair kit as defined above, wherein said prosthetic mesh device has configuration selected from a group consisting of a circular configuration, a rectangular configuration or any combination thereof.

It is another object of the present invention to provide a method for repairing a post operative ventral hernia. The method comprises steps selected inter alia from:
(a) providing a hernia repair kit, said kit comprising;
   (i) an umbrella-like device comprising a plurality of expandable arms 907 and a mesh 906 mounted to said arms 907; wherein said umbrella-like device is adapted for being advanced in a non-expanded state through said hernia defect using a trocar and expanded automatically upon release from said trocar;
   (ii) anchoring elements 22 at the distal ends of said arms 907, said anchoring elements 22 being adapted for switching from a closed configuration in which said anchoring elements 22 are embedded within said mesh 906, to an open configuration in which said anchoring elements 22 are protruding out of said mesh 906; said anchoring elements being adapted to secure said mesh 906 to said body wall 910;
   (iii) an expandable element 911 characterized by two configurations (i) unexpanded configuration in which said expandable element 911 is adapted to be pushed through said trocar and out of said trocar; (ii) expanded configuration in which said expandable element 911 is adapted to be expanded upon release from said trocar so as to (i) exert a normal force against said mesh 906 and arms 907 in the direction of said abdominal wall; and, (ii) secure said anchoring elements 22 to said abdominal wall 910;
(b) insufflating the abdominal wall;
(c) performing dissection of the bowel loops of the hernia;
(d) introducing said umbrella-like device to the hernia site using a trocar and opening said umbrella-like device inside of the inner abdominal wall;
(e) fixing said prosthetic device into place using said anchoring device, and;
(f) optionally, using a mesh patch to seal the hernia opening.

It is another object of the present invention to provide the method as defined above, additionally comprising step of selecting said expandable element 911 to be a balloon.

It is another object of the present invention to provide the method as defined above, additionally comprising step of selecting said anchoring elements from hooks, staple, tacks or any combination thereof.

It is another object of the present invention to provide the method as defined above, additionally comprising step of forming said prosthetic device from a material selected from a group consisting of Nitinol or electroactive polymers or any combination thereof.

It is another object of the present invention to provide the method as defined above, additionally comprising step of extending outward said arms of said anchoring device in a substantially radial manner

It is another object of the present invention to provide a hernia mesh 906 comprising a plurality of arms 907; said mesh is adapted for being advanced in a non-expanded state through said hernia defect using a trocar and expanded automatically upon release from said trocar;
(b) anchoring elements 22 at the distal ends of said arms 907, said anchoring elements 22 being adapted for switching from a closed configuration in which said anchoring elements 22 embedded within said mesh 906, to an open configuration in which said anchoring elements 22 are protruding out of said mesh 906; said anchoring elements being adapted to secure said mesh 906 to said abdominal wall 910.

It is another object of the present invention to provide the mesh as defined above, adapted for use in repairing a post operative ventral hernia.

It is another object of the present invention to provide the mesh as defined above, wherein said anchoring elements comprise elements selected from a group consisting of hooks, staple, tacks or any combination thereof.

It is another object of the present invention to provide the mesh as defined above, wherein said mesh is formed from material selected from a group consisting of Nitinol or electroactive polymers or any combination thereof.

It is another object of the present invention to provide the mesh as defined above, wherein arms of said mesh extend outward in a substantially radial manner.

It is another object of the present invention to provide the mesh as defined above, wherein said mesh has a configuration selected from a group consisting of a circular configuration, a rectangular configuration or any combination thereof.

It is another object of the present invention to provide the mesh as defined above, wherein said mesh 906 is further provided with additional anchoring elements.

It is lastly an object of the present invention to provide the mesh as defined above, wherein said anchoring elements 22 are made of a material having a spring-like characteristic of returning to an equilibrium configuration, said equilibrium configuration being adapted to allow attachment of said anchoring elements to said abdominal wall 910.

### BRIEF DESCRIPTION OF THE DRAWINGS

The present invention will now be described, by way of example only, with respect to the accompanying Figures, wherein:

FIG. 1 illustrates the formation of a POVH;

FIG. 2 illustrates the reduction of the loop to the abdominal cavity;

FIG. 3 illustrates the hernia defect after the bowel loops have been dissected therefrom, and the insertion of the trocar;

FIG. 4 illustrates the introduction of a prosthetic device through the trocar through the hernia defect;

FIG. 5 illustrates opening of the umbrella-like prosthetic device;

FIG. 6A1 illustrates introduction of an anchoring device through the trocar and opening the same;

FIG. 6A2 illustrates the prosthetic device pushed by the anchoring device;

FIG. 6A3 illustrates the device in place;

FIG. 7 illustrates the prosthetic device anchored to the abdominal wall, and;

FIGS. 8A, 8B and 8C illustrate possible configurations for the prosthetic device.

FIGS. 9A-R illustrate the method and system of the device, including insertion, deployment, and refraction phases.

FIGS. 10A,B illustrate one possible embodiment of the hook-like attachment elements of the device.

### DETAILED DESCRIPTION OF THE DRAWINGS

The description herein provides the reader with exemplary embodiments of the present invention and the methods for their usage. The description is, however, not meant to be limiting in any way, to the spirit and scope of the invention, as set out in the appended claims.

During the hernia repair procedure, a small incision is first made carefully through the hernia sac in order to allow for introduction of an 11 mm trocar. The abdominal wall then is insufflated. Alternatively, the trocar may be introduced laterally in the abdominal wall after insufflation of the abdominal cavity to 15 mm Hg pressure using a Verse needle.

Once the abdominal wall is insufflated, two other trocars are preferably introduced: one of 11 mm diameter, and one of 5 mm. Careful dissection of the bowel loops from the POVH is then performed.

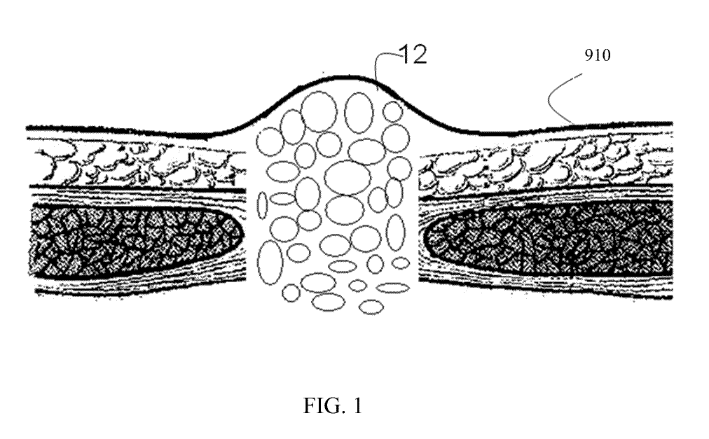

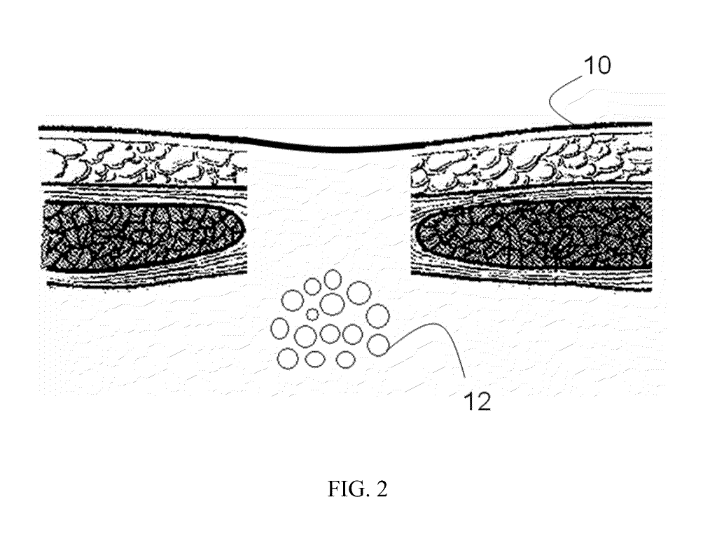

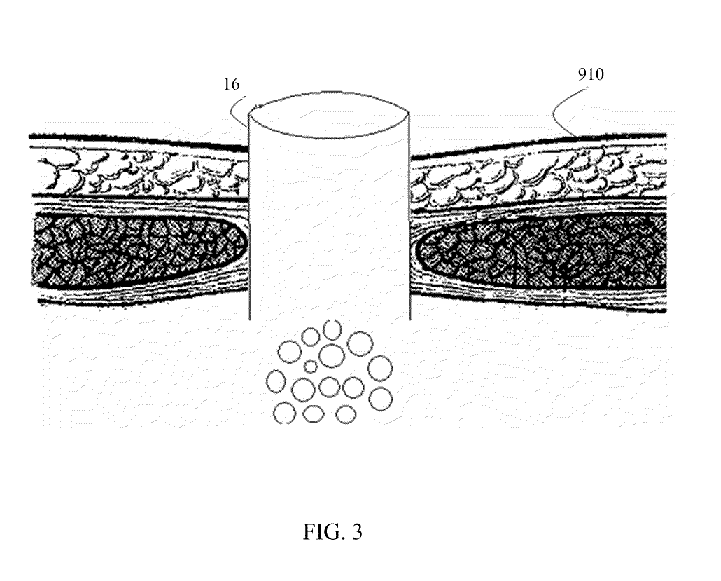

FIG. 1 illustrates a POVH in the abdominal wall (910). FIG. 1 shows that abdominal content (12) enters and protrudes through a defect(s) in the abdominal wall. In FIG. 2, the tissue has been dissected and the defect now requires closing by the surgeon. After the defect has been fully dissected, with at least 5-10 cm on each side, the prosthetic device (will be referred herein after as umbrella-like device) (14) of the hernia repair kit of the present invention is introduced through the trocar (16). The prosthetic device (14) is designed to open automatically upon release from trocar (16), due to the temperature of the body (see FIGS. 3-5), the springlike nature of the device arms, or other activation means as will be obvious to one skilled in the art.

The prosthetic device includes a mesh mounted on an expandable supporting frame, are well-known in the art. The prosthetic device may be formed, for example, from Nitinol, electroactive polymers and/or from another metal alloy exhibiting shape memory.

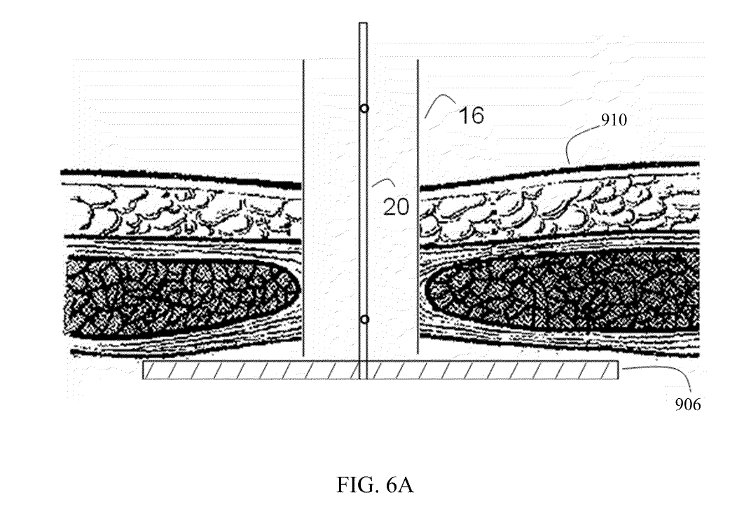

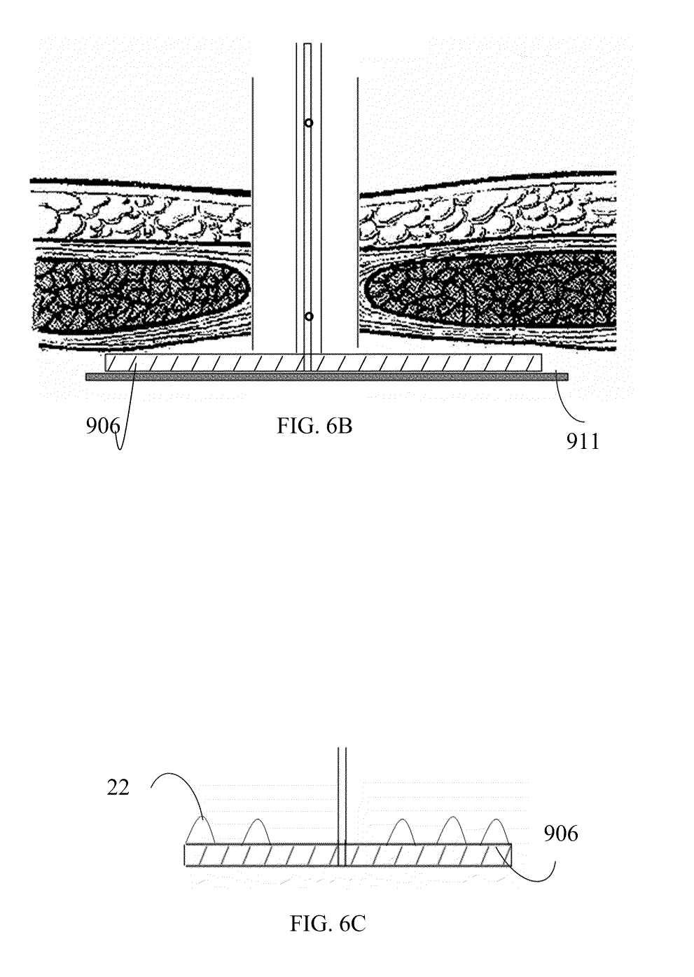

FIG. 6A illustrates introduction of an anchoring device through the trocar and opening the same; FIG. 6B illustrates the prosthetic device pushed by the expanding device; FIG. 6C illustrates the device in place.

The umbrella-like device (14) comprising a plurality of expandable arms 907 and a mesh 906 mounted to said arms 907. The umbrella-like device is adapted for being advanced in a non-expanded state through the hernia defect using a trocar and expanded automatically upon release from said trocar.

It should be noted that said arms 907 extend outwardly from said trocar in a substantially radial manner.

The kit additionally comprising anchoring elements 22 at the distal ends of said arms 907; said anchoring elements 22 being adapted for switching from a closed configuration in which said anchoring elements 22 are embedded within said mesh 906, to an open configuration in which said anchoring elements 22 are protruding out of said mesh 906; said anchoring elements being adapted to secure said mesh 906 to said abdominal wall 910

According to one embodiment of the present invention the anchoring elements comprise elements selected from a group consisting of hooks, staple, tacks or any combination thereof. According to one embodiment of the present invention the umbrella-like device is formed from material selected from a group consisting of Nitinol or electroactive polymers or any combination thereof.

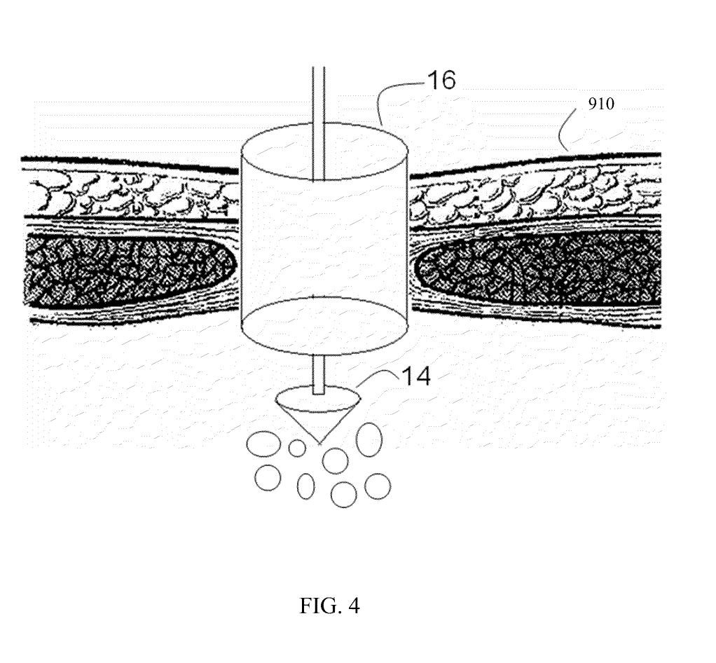

Reference is now made to FIG. 4 in which the umbrella-like device (14) had just been extracted from the trocar and is starting to transform from the non-expanded state to the expanded state.

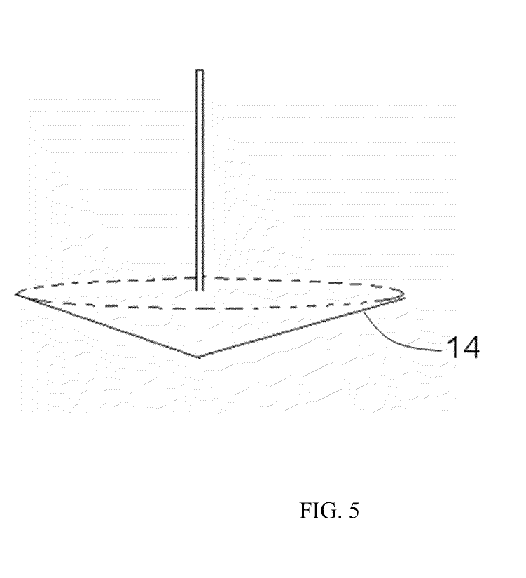

Reference is now made to FIG. 5 which illustrates the umbrella-like device (14) in its full expanded state.

reference is now made to FIGS. 6A-6C. Once the umbrella-like device (14) is expanded, a secondary component of the hernia repair kit is introduced, namely, the an expandable element 911. The expandable element 911 is characterized by two configurations (i) unexpanded configuration in which said expandable element 911 is adapted to be pushed through said trocar and out of said trocar; (ii) expanded configuration in which said expandable element 911 is adapted to be expanded upon release from said trocar so as to (i) exert a normal force against said mesh 906 and arms 907 in the direction of the abdominal wall; and, (ii) secure said anchoring elements 22 to said abdominal wall 910.

According to one embodiment said expandable element 911 is a balloon.

According to one embodiment of the present invention the expandable element 911 comprises a plurality of arms which extend outward radially from a central support rod (20). Like the prosthetic device (14), the arms are initially folded inside of the trocar (16). Once the end of the expandable element 911 is advanced from the end of the trocar (16) and through the prosthetic device, the arms automatically open up to extend outward radially. The surgeon then manipulates the expandable element 911 through the use of the support rod (20) such that arms of the expandable element 911 push the prosthetic device against the inside of the abdominal wall (10) (see FIGS. 6A-6C). At this stage, the hooks (22) of the prosthetic device (14) become affixed to the abdominal wall (10), such that the prosthetic mesh device (14) is now secured into place.

FIG. 6C illustrates the mesh 906 with the anchoring elements 22 (positioned in the distal ends of said arms 907) in its expanded state (once it had exited the trocar).

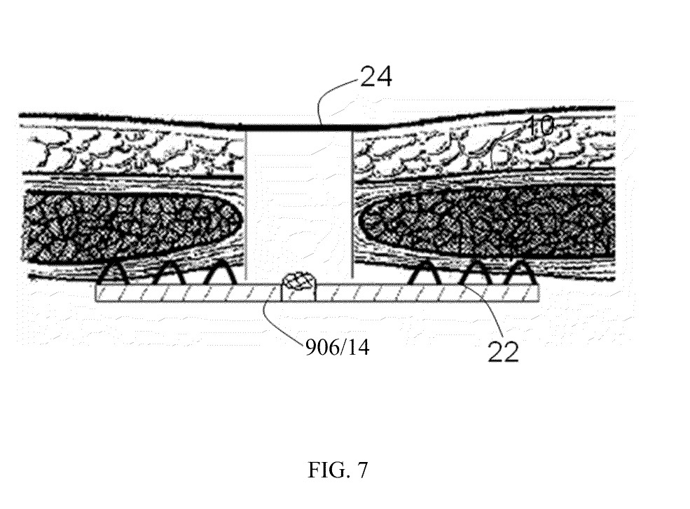

One the mesh is safely secured to the abdominal wall, the expanding device (906) is then collapsed into the trocar (16) and withdrawn from the body, leaving the prosthetic device in place (see FIG. 7). It will be appreciated that no other known hernia repair device of the prior art employs such an anchoring device in combination with an expanding device in order to facilitate anchoring of the prosthetic device into place.

Optionally, a mesh patch (24), or other suitable woven element, may be introduced over the hole or small incision in the hernia, as seen in FIG. 7.

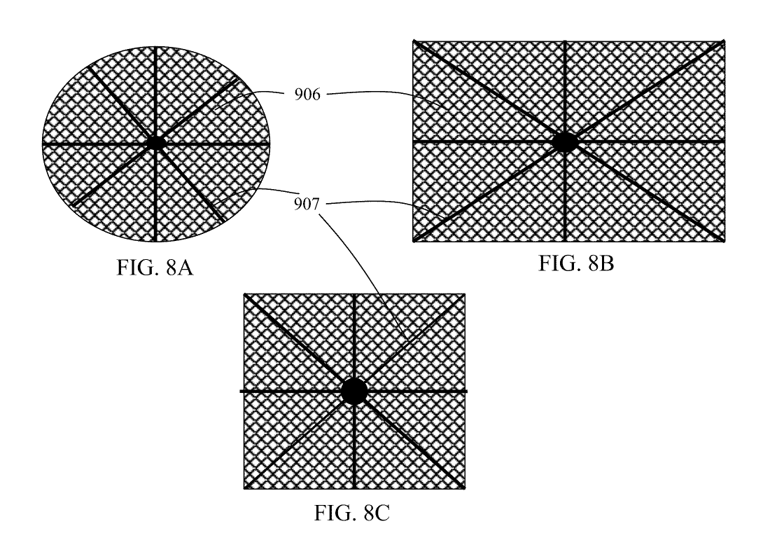

As appreciated by FIGS. 8A, 8B and 8C the prosthetic device may have any suitable shape, such as, but not limited to, a generally circular shape (FIG. 8A) or a generally rectangular shape (FIG. 8B, FIG. 8C). The surgeon may chose a prosthetic device having any suitable size and shape so as to cover the entire hernia defect, with an additional 5-10 cm all around. The mesh 906 and arms 907 of the umbrella-like prosthetic device are visible.

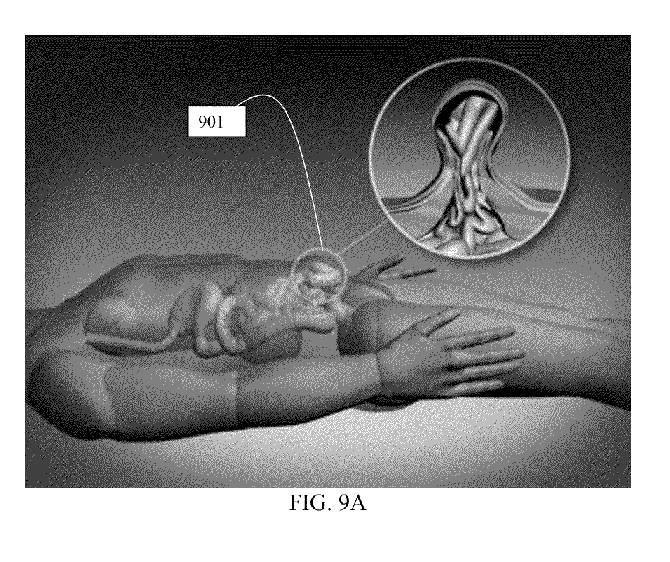

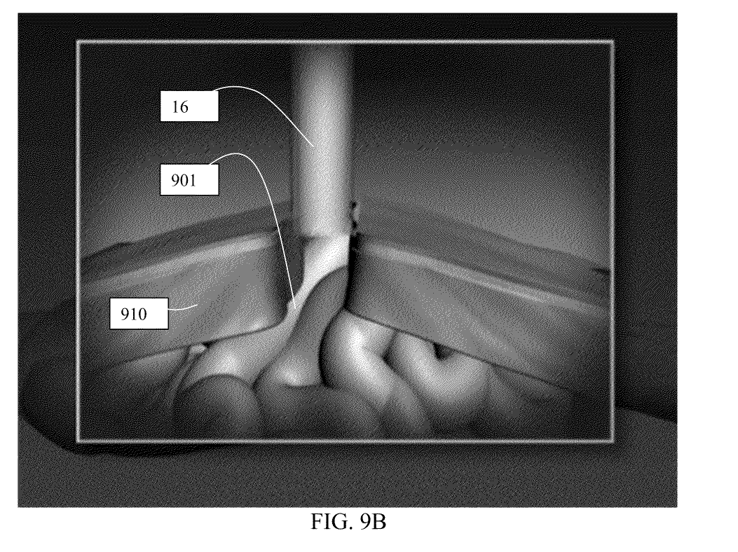

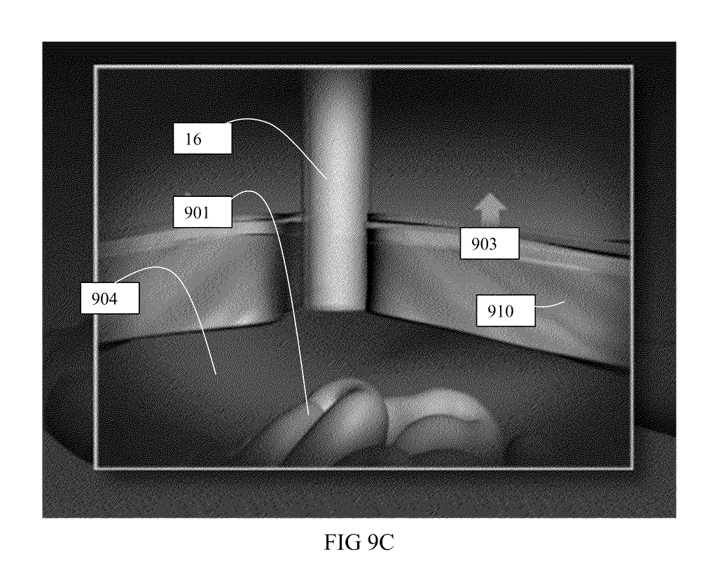

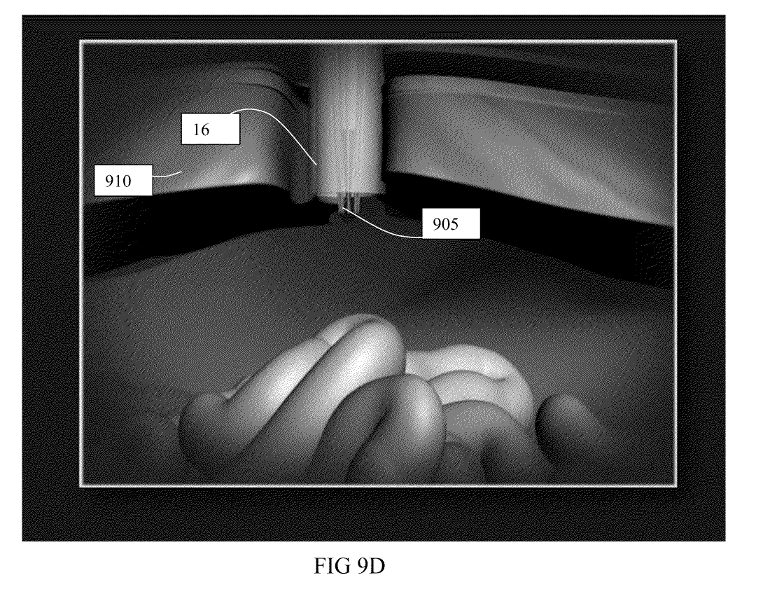

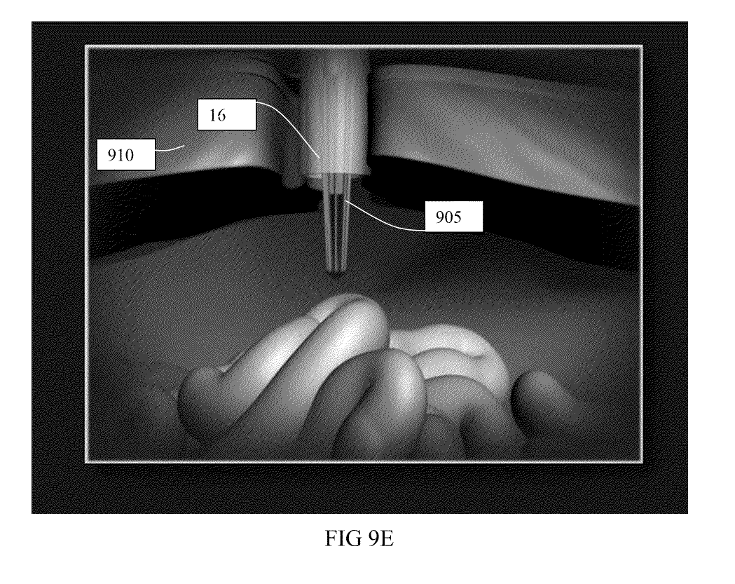

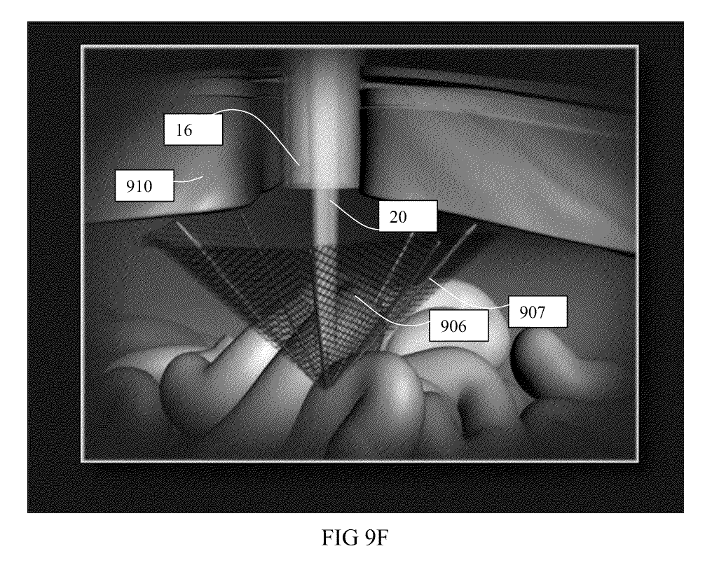

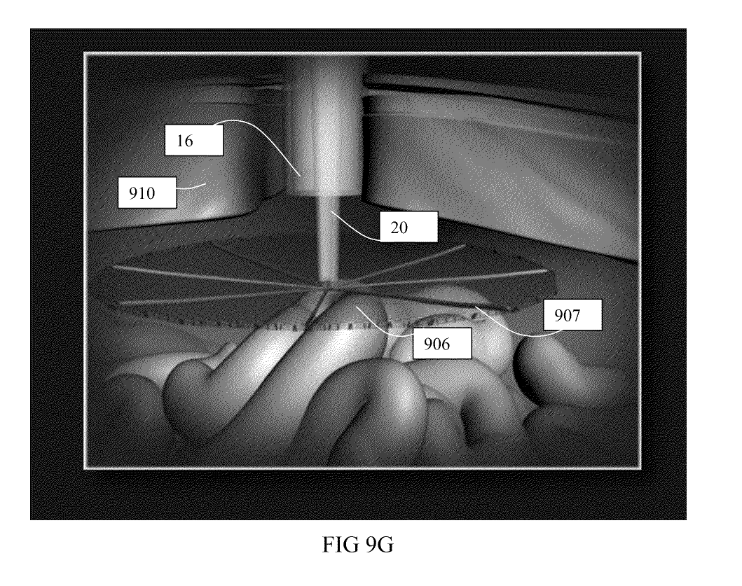

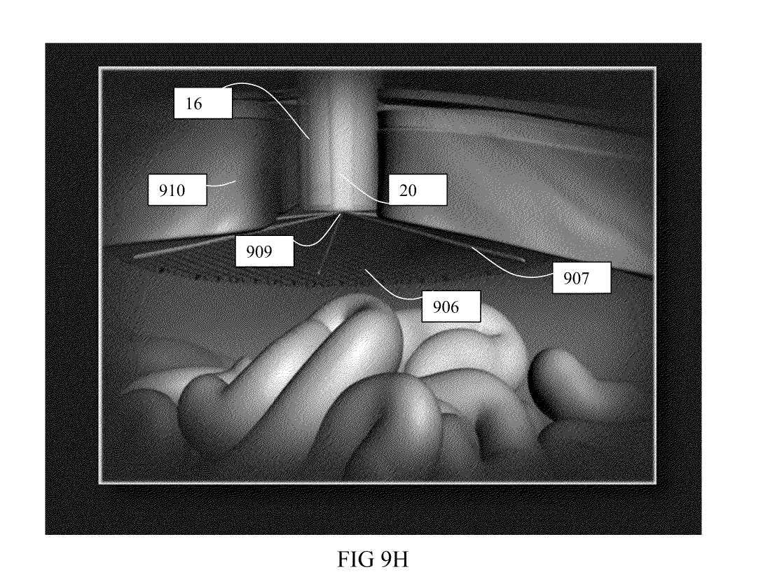

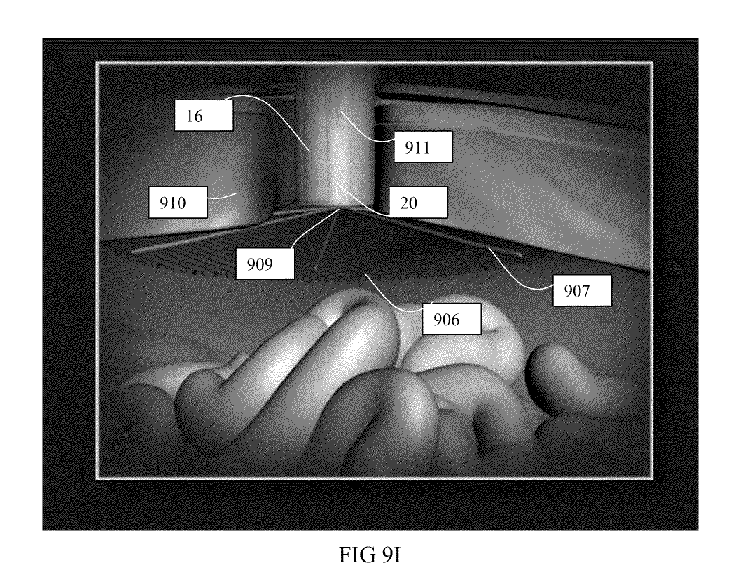

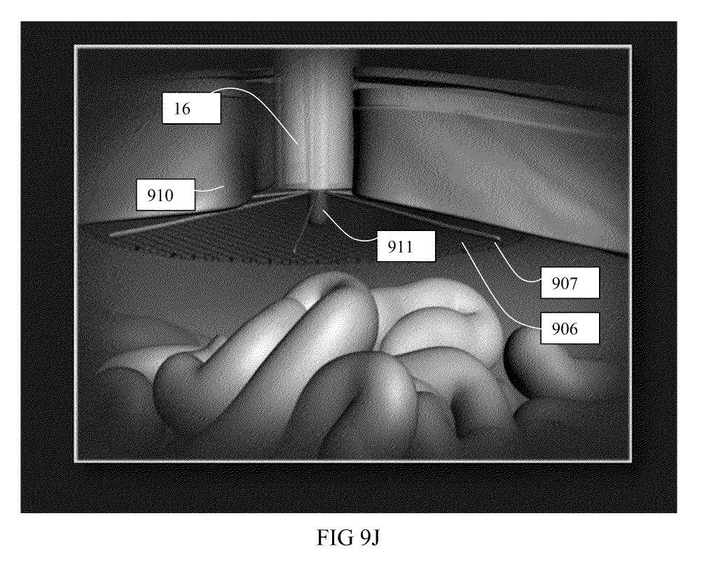

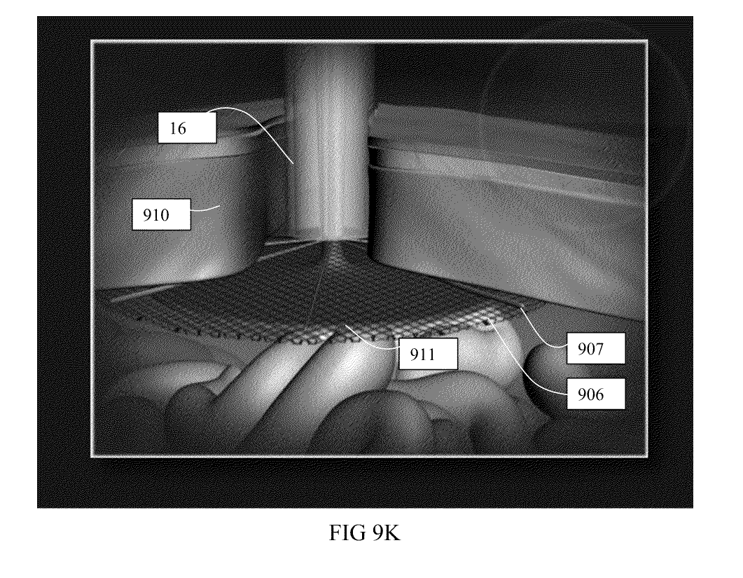

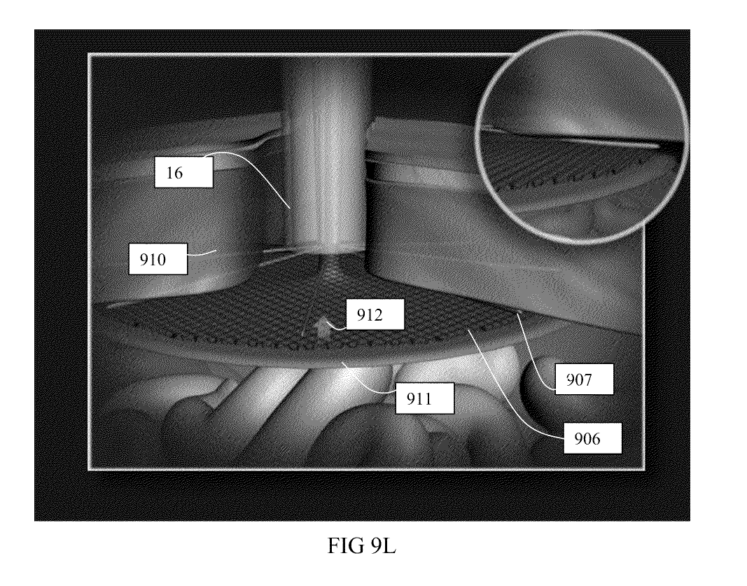

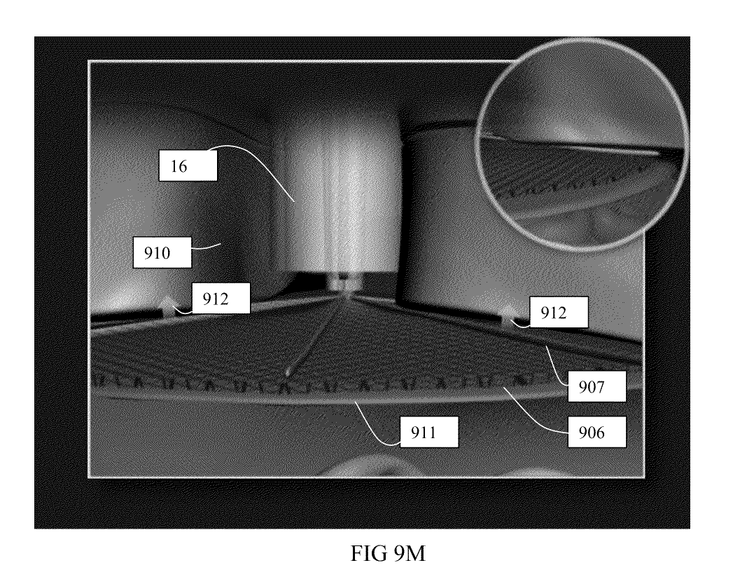

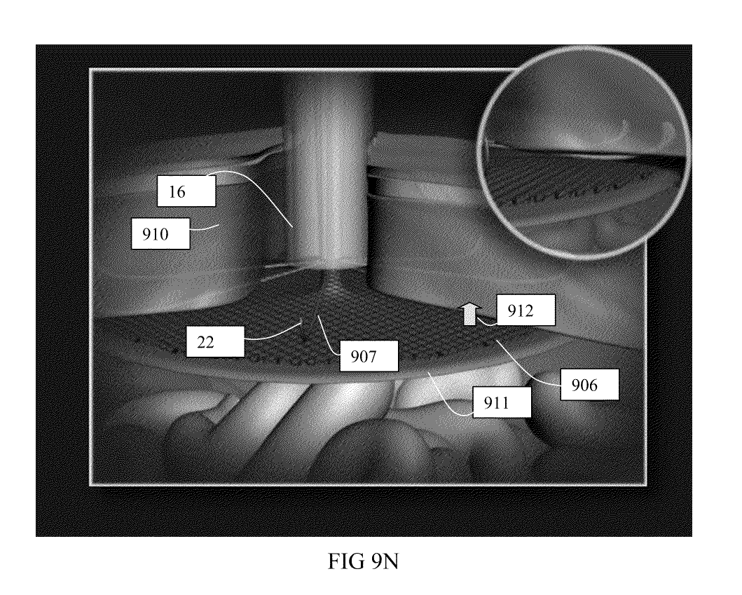

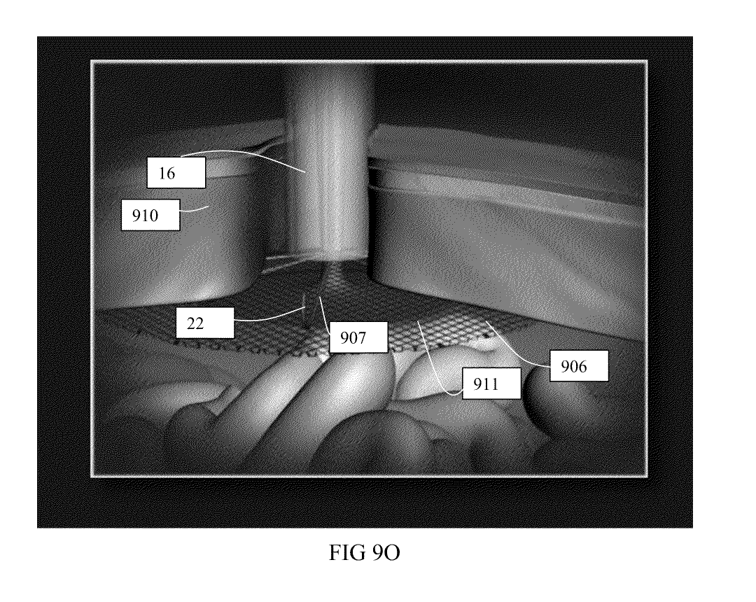

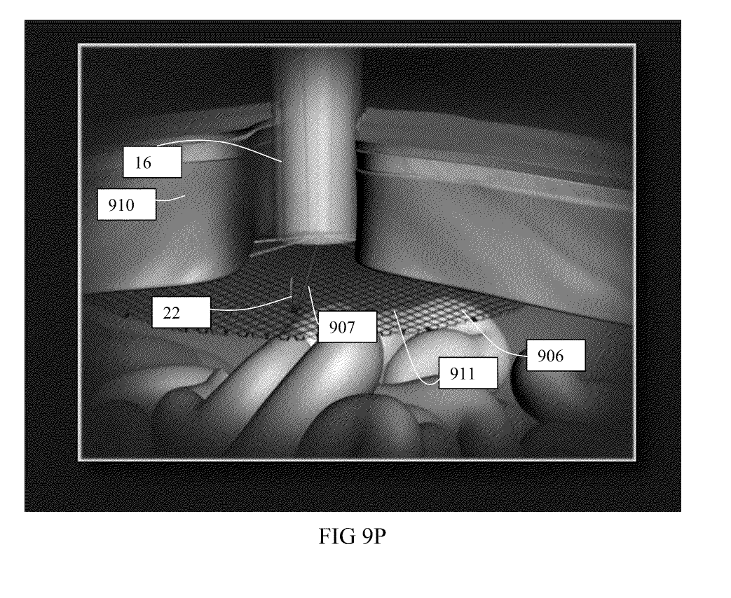

We now revisit the device and method of the invention with reference to a series of illustrative FIGS. 9A-9Q.

In FIG. 9A one sees a human body with hernia 901.

In FIG. 9B a closer view is seen of hernia 901, as well as the trocar 16 during insertion into an incision in the area of hernia 901. The abdominal wall 910 is seen in cross section.

In FIG. 9C the chest cavity 904 has been inflated with air, according to the method of the invention, thus pulling the abdomen in the direction 903 away from the herniated area.

In FIG. 9D the trocar 16 is seen as well as the umbrella-like expandable frame 905 of the invention, which consists of folded arms supporting a mesh. At this point in deployment the umbrella-like device is still within the trocar and therefore it is folded, undeployed, and the mesh is hidden from view.

In FIG. 9E the umbrella-like device 905 has been further inserted into the chest cavity through the trocar, exposing more of the device arms.

In FIG. 9F one sees the umbrella-like device which in this particular embodiment comprises mesh 906, arms 907 and central support rod or shaft 20. The umbrella-like device has partially unfolded due to the spring-like characteristic of arms 907, which have an equilibrium position perpendicular to the shaft 20. When folded inside the trocar 16, the arms 907 are under compression, and will tend to spring open when released from the confines of the trocar. Other embodiments adapted to deploy the arms 907 are possible as will be obvious to one skilled in the art.

In FIG. 9G the umbrella-like device has fully unfolded. One sees the trocar 16, the shaft 20, the arms 907, and the mesh 906 which is now pulled taut by the arms 907, which are now in their fully deployed position.

In FIG. 9H the shaft 20 is pulled partially out of the chest cavity, pulling the junction 909 at which all the arms 907 meet away from the chest cavity and towards the abdomen wall 910.

In FIG. 9I an inflatable balloon-like element 911 is shown during insertion through the trocar.

In FIG. 9J the balloon-like element 911 is seen protruding into the chest cavity, having been further pushed distally into the trocar.

In FIG. 9J the balloon-like element 911 is seen during inflation. In the particular embodiment shown here, the inflated balloon takes a disc-like or pancake-like form, parallel to the mesh 906. Other embodiments are possible as will be obvious to one skilled in the art, such as a spherical or ovoid balloon.

In FIG. 9L one sees the balloon-like element after full inflation, now extending past the mesh 906. The balloon applies a force in the direction 912 away from the chest cavity and towards the abdomen wall due to its inflation.

In FIG. 9M a closer view is seen which more clearly shows the outward force 912 applied by the balloon upon the mesh 906 and arms 907 of the device.

In FIG. 9N the next stage of deployment is seen in which the arms 907 are caused to form hooks 2222 at their ends. This may be accomplished by means that will be obvious to one skilled in the art, such as the employment of arms having two halves, which are pulled one relative to the other to achieve a bending moment. The position of greatest bend can be controlled by changing the thickness of the arms, the greatest bending occurring in the region of least thickness. The bent hook-like ends 22 of the arms 907 are effectively driven into the tissue 910 of the abdomen by means of the upwards pressure 912 applied by the balloon-like device 911.

In FIG. 9O the balloon-like device 911 is deflated somewhat, to allow it to be withdrawn back into the trocar 16.

In FIG. 9P the balloon-like device has been entirely withdrawn back into the trocar 16, leaving the mesh 911 affixed into place against the abdomen wall 910 by means of the arms 907, which attach to the abdomen wall 910 by their hook-like ends 22.

In FIG. 9Q a view from with the chest cavity is seen of the mesh 911 which is held by arms 907 that attach to the abdomen wall by means of their hook-like ends 22. A side view of the same situation is shown in FIG. 9R.

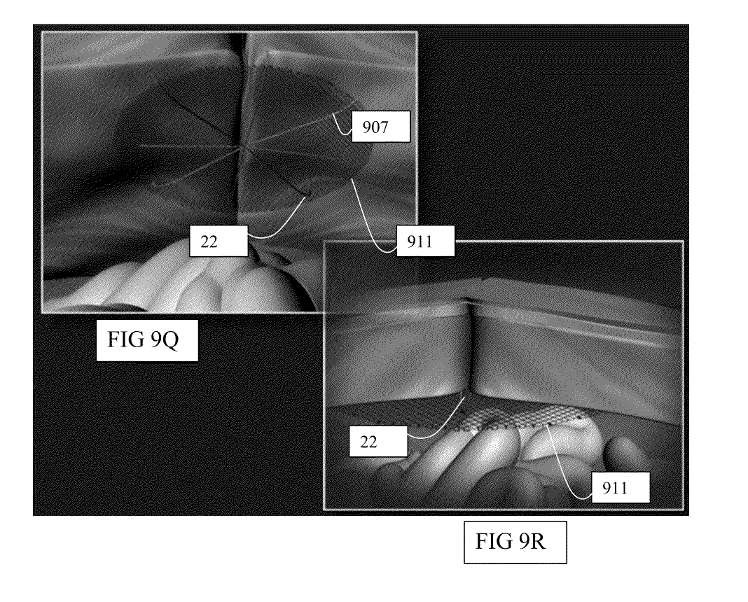

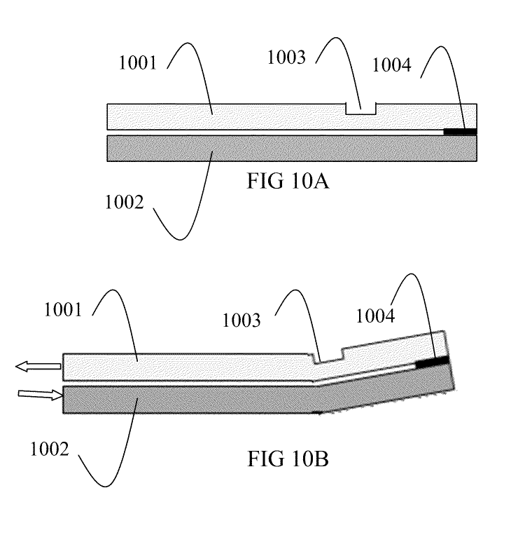

In FIG. 10 one possible deployment means for the hook-like elements 22 at the ends of arms 907 of FIG. 9 is shown. The arms 907 (FIG. 9) are actually composed of dual strips 1001,1002 that are attached to one another at the distal ends 1004. One of the strips 1001 has a thinner section 1003 that will tend to more easily allow deformation than the remainder of the strips due to its thinner and hence weaker cross section. When the strips are pulled relative to one another in the directions indicate by arrows (FIG. 10B), the arm will tend to pull upward as shown due to deformation at or near point 1003.

1. A hernia repair kit adapted to repair hernia defect by attachment of a mesh to the abdominal wall, said kit comprising:
(a) an umbrella-like device (14) comprising a plurality of expandable arms 907 and a mesh 906 mounted to said arms 907; wherein said umbrella-like device is adapted for being advanced in a non-expanded state through said hernia defect using a trocar and expanded automatically upon release from said trocar;
(b) anchoring elements 22 at the distal ends of said arms 907, said anchoring elements 22 being adapted for switching from a closed configuration in which said anchoring elements 22 are embedded within said mesh 906, to an open configuration in which said anchoring elements 22 are protruding out of said mesh 906; said anchoring elements being adapted to secure said mesh 906 to said abdominal wall 910;
(c) an expandable element 911 characterized by two configurations (i) unexpanded configuration in which said expandable element 911 is adapted to be pushed through said trocar and out of said trocar; (ii) expanded configuration in which said expandable element 911 is adapted to be expanded upon release from said trocar so as to (i) exert a normal force against said mesh 906 and arms 907 in the direction of said abdominal wall; and, (ii) secure said anchoring elements 22 to said abdominal wall 910.

2. The hernia repair kit of claim 1, wherein said expandable element 911 is a balloon.

3. The hernia repair kit of claim 1, adapted for use in repairing a post operative ventral hernia.

4. The hernia repair kit of claim 1, wherein said anchoring elements comprise elements selected from a group consisting of hooks, staple, tacks or any combination thereof.

5. The hernia repair kit of claim 1, wherein said device is formed from material selected from a group consisting of Nitinol or electroactive polymers or any combination thereof.

6. The hernia repair kit of claim 1, wherein said arms extend outward in a substantially radial manner.

7. The hernia repair kit of claim 1, wherein said prosthetic mesh device has configuration selected from a group consisting of a circular configuration, a rectangular configuration or any combination thereof.

8. A method for repairing a post operative ventral hernia, comprising;
(a) providing a hernia repair kit, said kit comprising;
(i) an umbrella-like device (14) comprising a plurality of expandable arms 907 and a mesh 906 mounted to said arms 907; wherein said umbrella-like device is adapted for being advanced in a non-expanded state through said hernia defect using a trocar and expanded automatically upon release from said trocar;
(ii) anchoring elements 22 at the distal ends of said arms 907, said anchoring elements 22 being adapted for switching from a closed configuration in which said anchoring elements 22 are embedded within said mesh 906, to an open configuration in which said anchoring elements 22 are protruding out of said mesh 906; said anchoring elements being adapted to secure said mesh 906 to said body wall 910;
(iii) an expandable element 911 characterized by two configurations (i) unexpanded configuration in which said expandable element 911 is adapted to be pushed through said trocar and out of said trocar; (ii) expanded configuration in which said expandable element 911 is adapted to be expanded upon release from said trocar so as to (i) exert a normal force against said mesh 906 and arms 907 in the direction of said abdominal wall; and, (ii) secure said anchoring elements 22 to said abdominal wall 910;
(b) insufflating the abdominal wall;
(c) performing dissection of the bowel loops of the hernia;
(d) introducing said umbrella-like device to the hernia site using a trocar and opening said umbrella-like device inside of the inner abdominal wall;
(e) fixing said prosthetic device into place using said anchoring device, and;
(f) optionally, using a mesh patch to seal the hernia opening.

9. The method according to claim 8, additionally comprising step of selecting said expandable element 911 to be a balloon.

10. The method according to claim 8, additionally comprising step of selecting said anchoring elements from hooks, staple, tacks or any combination thereof.

11. The method according to claim 8, additionally comprising step of forming said device from a material selected from a group consisting of Nitinol or electroactive polymers or any combination thereof.

12. The method according to claim 8, additionally comprising step of extending outward said arms of said anchoring device in a substantially radial manner

13. A hernia mesh 906 comprising a plurality of arms 907; said mesh is adapted for being advanced in a non-expanded state through said hernia defect using a trocar and expanded automatically upon release from said trocar;
(b) anchoring elements 22 at the distal ends of said arms 907, said anchoring elements 22 being adapted for switching from a closed configuration in which said anchoring elements 22 embedded within said mesh 906, to an open configuration in which said anchoring elements 22 are protruding out of said mesh 906; said anchoring elements being adapted to secure said mesh 906 to said abdominal wall 910.

14. The hernia mesh of claim 13, adapted for use in repairing a post operative ventral hernia.

15. The hernia mesh of claim 13, wherein said anchoring elements comprise elements selected from a group consisting of hooks hooks, staple, tacks or any combination thereof.

16. The hernia mesh of claim 13, wherein said mesh is formed from material selected from a group consisting of Nitinol or electroactive polymers or any combination thereof.

17. The hernia mesh of claim 13, wherein said arms extend outward in a substantially radial manner.

18. The hernia mesh of claim 13, wherein said mesh has a configuration selected from a group consisting of a circular configuration, a rectangular configuration or any combination thereof.

19. The hernia mesh of claim 13, wherein said mesh 906 is further provided with additional anchoring elements.

20. The hernia mesh of claim 9, wherein said anchoring elements 22 are made of a material having a spring-like characteristic of returning to an equilibrium configuration, said equilibrium configuration being adapted to allow attachment of said anchoring elements to said abdominal wall 910.
